(19) United States
(12) Patent Application Publication
(10) Pub. No.: US 2021/0128194 A1
(43) Pub. Date: May 6, 2021

(54) CHEST TUBE INTRODUCER

(71) Applicant: THE TRUSTEES OF THE UNIVERSITY OF PENNSYLVANIA, Philadelphia, PA (US)

(72) Inventor: Shariq Raza, Philadelphia, PA (US)

(73) Assignee: THE TRUSTEES OF THE UNIVERSITY OF PENNSYLVANIA, Philadelphia, PA (US)

(21) Appl. No.: 17/085,839

(22) Filed: Oct. 30, 2020

Related U.S. Application Data
(60) Provisional application No. 62/928,042, filed on Oct. 30, 2019.

(51) Int. Cl. A61B 17/34

(52) U.S. Cl. CPC A61B 17/3415; A61B 17/3417

(57) ABSTRACT

A chest tube introducer is disclosed. In some embodiments, the introducer includes a tubular shaft adapted for insertion into a pleural cavity and through which a chest tube can be threaded, and a gripping mechanism that is coupled to a proximal end of the shaft and adapted for positioning the chest tube introducer upon insertion of the shaft into the pleural cavity. The introducer can also include an obturator removably coupled to a distal end of the shaft such that the obturator can be removed from the shaft after insertion thereof into the pleural cavity, and an outer flange that is coupled to the obturator and adapted to remove the obturator from the shaft.

### CROSS-REFERENCE TO RELATED APPLICATION

This application claims priority to U.S. Provisional Patent Application No. 62/928,042, filed Oct. 30, 2019, the contents of which are incorporated by reference herein in its entirety.

### BACKGROUND

Certain techniques for air or fluid or blood drainage in the pleural cavity are known. Such techniques include placement of a chest tube into the pleural cavity. An ideally placed chest tube is directed to the apex of the lung to evacuate air or to the base to evacuate fluid or blood. This position is often not achieved, however, due to the blind motion of the chest tube once it is introduced inside the thoracic cavity. Kelly clamps can be used to direct the chest tube, but the attendant directing effect can be limited to the incision site. As a result, such techniques may necessitate the placement of additional chest tubes with the same technical constraints or even additional surgery to remove retained blood clots, thereby leading to increased procedures, morbidity and mortality, and costs.

Accordingly, there exists a need for a technique for better directing chest tubes inside the thoracic cavity during placement.

### SUMMARY

The present disclosure provides a chest tube introducer. The present disclosure also discloses methods of inserting a chest tube into a pleural cavity.

In some embodiments, the introducer includes a tubular shaft adapted for insertion into a pleural cavity and through which a chest tube can be threaded. The introducer also includes a gripping mechanism coupled to a proximal end of the shaft and adapted for positioning the chest tube introducer upon insertion of the shaft into the pleural cavity.

In certain embodiments, the introducer includes an obturator removably coupled to a distal end of the shaft such that the obturator can be removed from the shaft after insertion thereof into the pleural cavity, and an outer flange coupled to the obturator and adapted to remove the obturator from the shaft. The chest tube introducer can be used to direct tubes to drain pneumothorax, hemothorax, hemo-pneumothorax, retained hemothorax, pleural effusions, loculated pleural effusions or collections.

In some embodiments, the disclosed subject matter can include an L-shaped tubular shaft. In some embodiments, the disclosed subject matter can include two grip holes as the gripping mechanism. In some embodiments, the disclosed subject matter can include a tubular shaft with at least one aperture. In some embodiments, the disclosed subject matter can include a notch at the distal end of the obturator.

The accompanying drawings, which are incorporated and constitute part of this disclosure, illustrate preferred embodiments of the invention and serve to explain the principles of the disclosed subject matter.

### BRIEF DESCRIPTION OF THE DRAWINGS

FIG. 1 illustrates a perspective view of a chest tube introducer in accordance with an exemplary embodiment of the disclosed subject matter.

FIG. 2 illustrates a side view of a chest tube introducer in accordance with an exemplary embodiment of the disclosed subject matter.

FIG. 3 illustrates a front view of a chest tube introducer in accordance with an exemplary embodiment of the disclosed subject matter.

FIG. 4 illustrates a top view of a chest tube introducer in accordance with an exemplary embodiment of the disclosed subject matter.

FIG. 5 illustrates an enlarged view of the notched end of an obturator in accordance with an exemplary embodiment of the disclosed subject matter.

FIG. 6 illustrates a perspective view of a chest tube introducer without an obturator or an outer flange in accordance with an exemplary embodiment of the disclosed subject matter.

FIGS. 7-9 illustrate an alternative exemplary embodiment of a chest tube introducer with an obturator at various positions.

FIG. 10 shows a photograph of an exemplary chest tube introducer with an obturator according to certain embodiments.

FIG. 11 shows a photograph of an exemplary chest tube introducer and obturator separately.

FIG. 12 illustrates an exemplary embodiment of a chest tube introducer and provides exemplary dimensions.

FIG. 13 illustrates an exemplary embodiment of a chest tube introducer and provides exemplary dimensions.

Throughout the drawings, the same reference numerals and characters, unless otherwise stated, are used to denote like features, elements, components or portions of the illustrated embodiments. Moreover, while the present invention will now be described in detail with reference to the Figs., it is done so in connection with the illustrative embodiments.

### DETAILED DESCRIPTION

Techniques for directing a chest tube in the thoracic cavity during placement are presented. In an exemplary embodiment, the tubular shaft of the chest tube introducer enters and advances into the pleural cavity through a skin incision. Once the shaft is inserted into the pleural cavity, the obturator is removed from the shaft by pulling the outer flange. A chest tube is then threaded through the tubular shaft and advanced into the pleural cavity. The gripping mechanism is used to direct the chest tube along the inside of the chest wall toward the apex of the lung to evacuate air or to the base or another direction to evacuate blood or fluid collection.

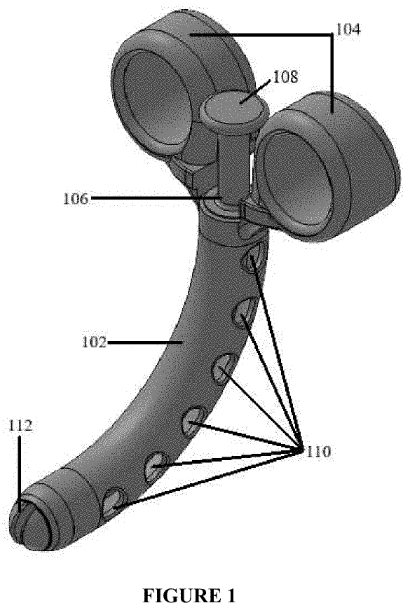

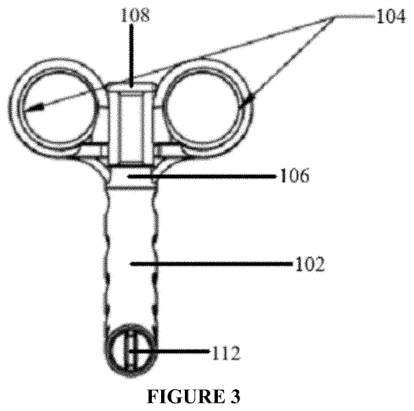

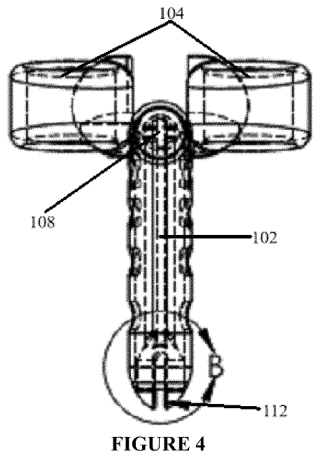

FIG. 1-4 illustrate different views of an example chest tube introducer in accordance with an embodiment of the disclosed subject matter. The chest tube introducer includes a tubular shaft 102, a gripping mechanism, an obturator 106, and an outer flange 108.

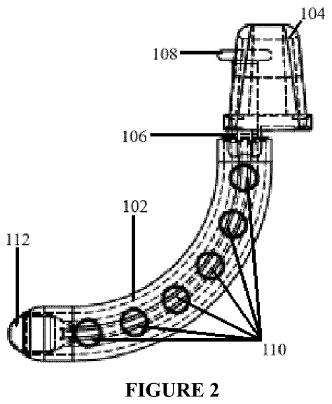

The tubular shaft 102 is adapted for insertion into the pleural cavity. In some embodiments, the tubular shaft 102 may be L-shaped or curved as shown in FIG. 1-2. An L-shaped or curved shape aligns the gripping mechanism when positioning the chest tube. In some embodiments, the tubular shaft contains at least one aperture 110 as shown in FIG. 1-2. The aperture(s) allows air or fluid or blood to flow through the shaft 102 and prevents clotting within the shaft.

The gripping mechanism is coupled to the proximal end of the tubular shaft 104 and adapted for positioning the chest tube introducer upon insertion of the distal end of the tubular shaft 104 into the pleural cavity. The coupling of the gripping mechanism to the proximal end of the shaft 104 allows the positioning to occur closer to the chest wall. In some embodiments, the gripping mechanism is two grip holes 104 as shown in FIG. 1-4. Each grip hole 104 is grasped to adjust the position of the chest tube introducer.

The obturator 106 is removably coupled to the distal end of the tubular shaft 104 such that the obturator 106 can be removed from the shaft 104 after insertion thereof into the pleural cavity. The obturator 104 fills the shaft 102 during insertion into the pleural cavity. In some embodiments, the obturator 104 is notched 112 at its distal end as shown in FIG. 1-4.

The outer flange 108 is coupled to the obturator 106 and adapted to remove the obturator 106 from the tubular shaft 102. Once the shaft 102 is inserted into the pleural cavity, the outer flange 108 allows the obturator 106 to be removed from the shaft 102.

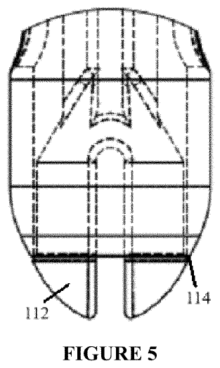

FIG. 5 illustrates an enlarged view of the notched end of an obturator in accordance with an embodiment of the disclosed subject matter. The notched end 112 enables the obturator to expand and snugly fit at the distal end of the shaft 102. The wedge 114 allows for and serves to reduce the distal end of the shaft 102 into a gradual and less obtrusive angle during insertion.

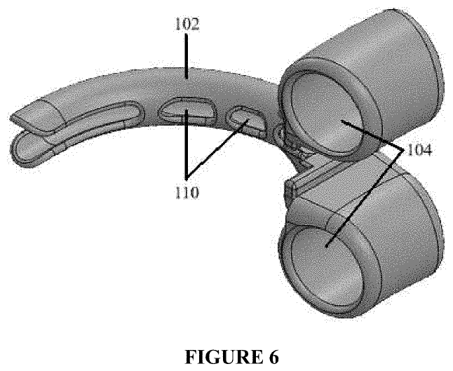

FIG. 6 illustrates a perspective view of a chest tube introducer without an obturator or an outer flange in accordance with an embodiment of the disclosed subject matter. The chest tube introducer includes a tubular shaft 102, two grip holes 104, and at least one aperture 110. After the outer flange and obturator are removed from the shaft 102, a chest tube can be threaded through the shaft 102. Once the chest tube is threaded through the shaft 102, the grip holes 104 can be used to position the chest tube introducer so that the chest tube is directed along the inside of the chest wall toward the apex of the lung to evacuate air or to the base or another direction to evacuate blood or fluid collection.

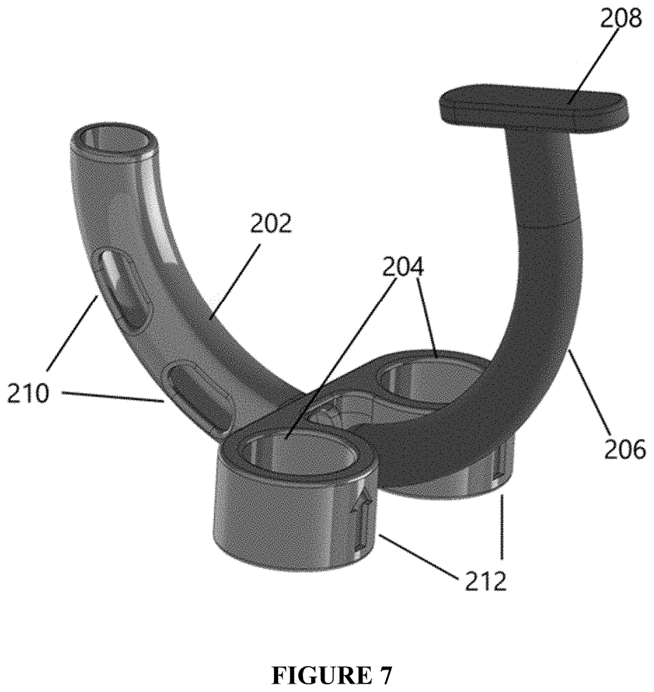

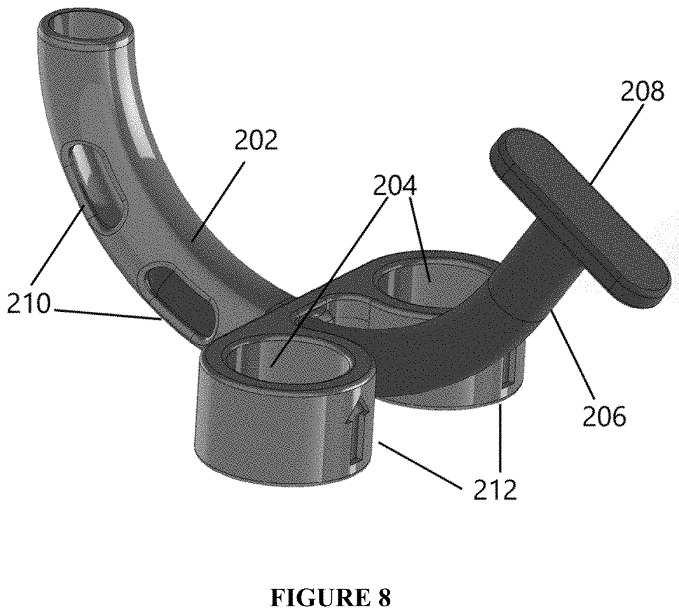

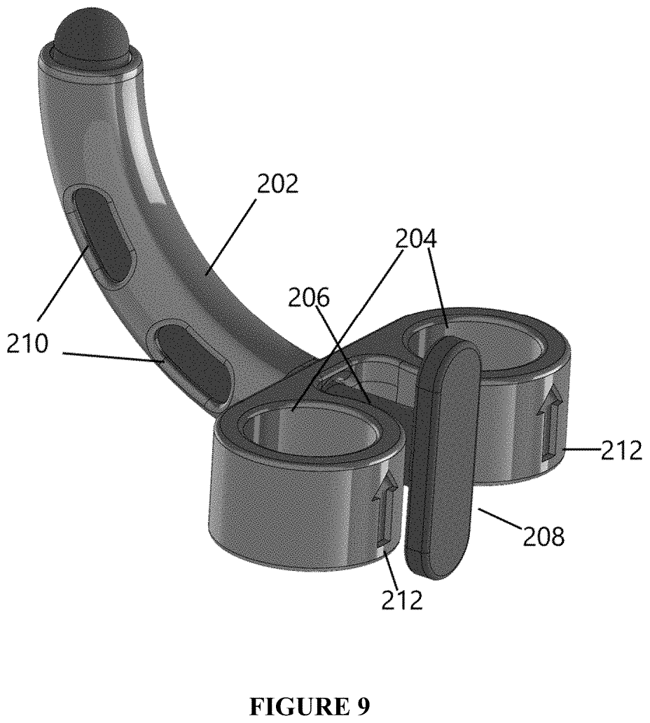

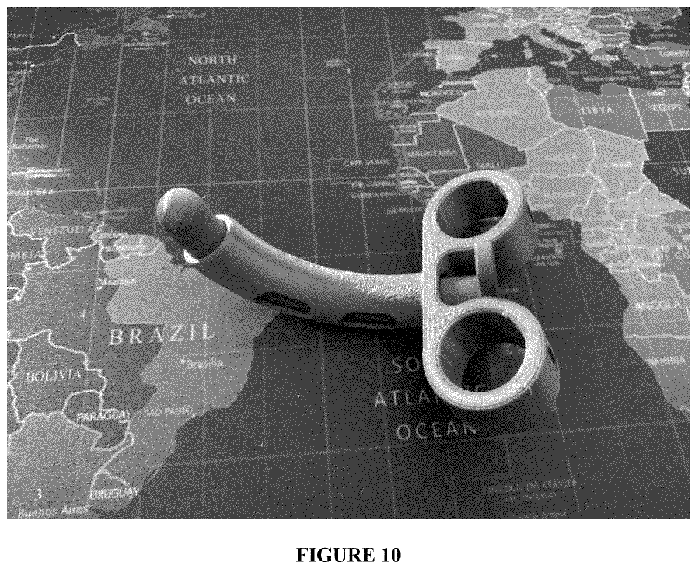

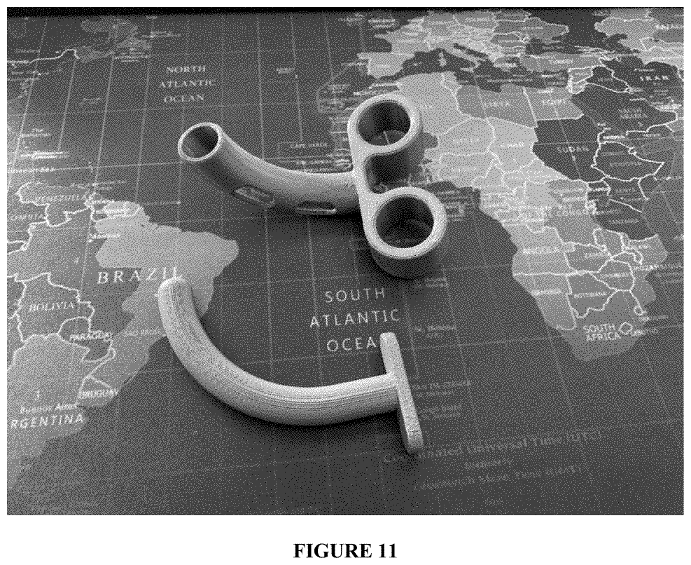

FIGS. 7-9 illustrate different views of an example chest tube introducer in accordance with an alternative embodiment of the disclosed subject matter. The chest tube introducer includes a tubular shaft 202, a gripping mechanism having two grip holes 204, an obturator 206, an outer flange 208 and at least one aperture 210. The aperture(s) allows air or fluid or blood to flow through the shaft 202 and prevents clotting within the shaft. The outer flange 208 is flattened to allow for stabilization during insertion of device into chest.

In certain embodiments, as show in FIGS. 7-9, the chest tube introducer can further include arrows 212 on the gripping mechanism that depict direction of introducer channel. The arrows enable a user to determine the direction of the introducer channel even when completely inserted inside chest cavity.

The chest tube introducer as shown in FIGS. 7-9 have has a shorter overall length of the curved portion of the introducer channel which now allows for and the curve has been adjusted for less restrictive flow of the obturator within the channel. In certain embodiments, the length of the curved portion of the introducer channel is from about 80 mm to about 90 mm, or about 85 mm.

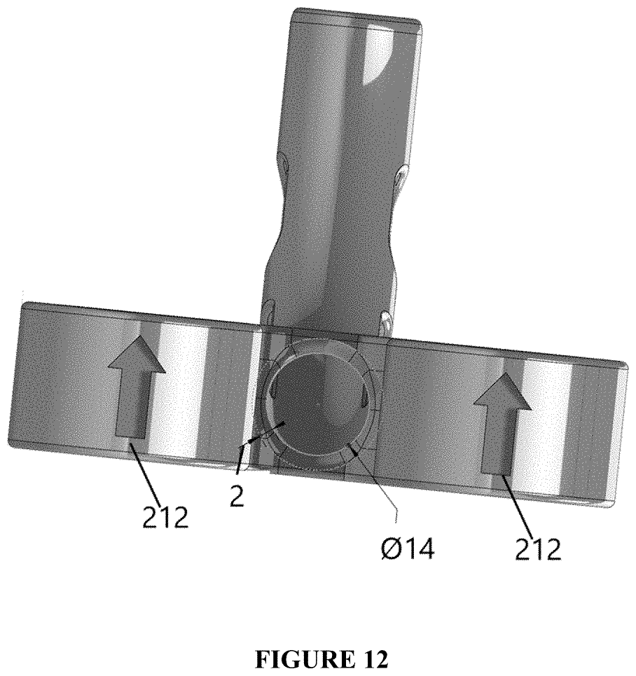

In certain embodiments, the inner diameter of the tubular shaft 202 is from about 10 mm to about 20 mm, from about 10 mm to about 15 mm, or from about 12 mm to about 16 mm. In certain embodiments, the inner diameter of the tubular shaft 202 is about 10 mm, about 11 mm, about 12 mm, about 13 mm, about 14 mm, about 15 mm, about 16 mm, about 17 mm, about 18 mm, about 19 mm or about 20 mm. In certain particular embodiments, as shown in FIG. 12,

In certain embodiments, the wall thickness of the tubular shaft 202 is about 2 mm, as shown in FIG. 12, the inner diameter of the tubular shaft 202 is 14 mm.

In certain embodiments, inner diameter of each of the grip holes is from about 20 to about 25 mm. In certain particular embodiments, the inner diameter of each of the grip holes is about 23 mm.

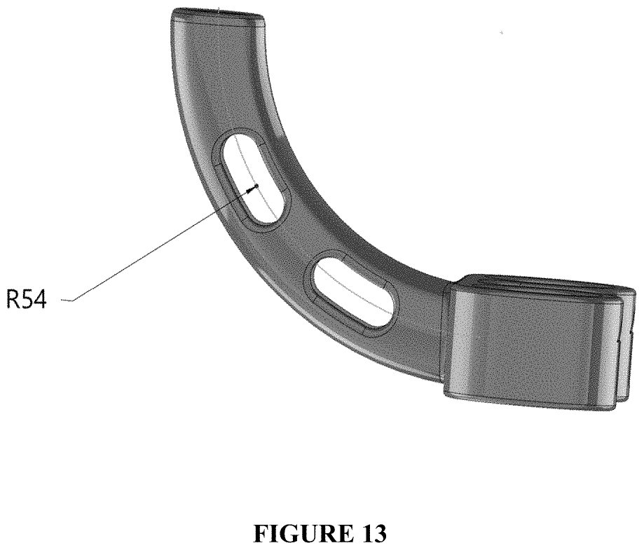

In certain embodiments, as shown in FIG. 13, the radius of the sweep (the curved tubular shaft) is about 54 mm.

### Examples

The chest tube introducer of the present disclosure was tested in a frozen cadaveric model and the results were compared to the traditional method. Results were tabulated using thoracotomy for verification of final chest tube landing position. The results are shown in Table 1.

TABLE 1

| | Traditional | Chest Tube Inserter of Present |
|---|---|---|
| | method (n = 8) | Disclosure (n = 7) |
| Achievement of intended | 63% | 86% |
| Apical placement | | |
| Achievement of intended | 50% | 57% |
| Anterior or Posterior | | |
| placement | | |

Complications

| | | |
|---|---|---|
| Kinked chest tube | 13% | 0% |
| Chest tube in lung fissure | 38% | 14% |

As shown in Table 1, the chest tube inserter of present disclosure achieved higher percentage of intended apical placement (86% vs. 63%), and a higher percentage of intended anterior or posterior placement (57% vs. 50%). Furthermore, fewer complications were seen with the chest tube inserter of present disclosure. Particularly, the when using the chest tube inserter of present disclosure, no kinked chest tubes were observed (while it was seen in 13% of the cases using traditional method) and the incidence of chest tube in lung fissure was lower (14% vs 38%).

The foregoing merely illustrates the principles of the disclosed subject matter. Various modifications and alterations to the described embodiments will be apparent to those skilled in the art in view of the teachings herein. It will thus be appreciated that those skilled in the art will be able to devise numerous techniques which, although not explicitly described herein, embody the principles of the disclosed subject matter and are thus within its spirit and scope.

1. A chest tube introducer comprising:
a tubular shaft adapted for insertion into a pleural cavity, wherein a chest tube can be threaded through the shaft;
a gripping mechanism, coupled to a proximal end of the shaft, adapted for positioning the chest tube introducer upon insertion of the shaft into the pleural cavity;
an obturator removably coupled to a distal end of the shaft such that the obturator can be removed from the shaft after insertion thereof into the pleural cavity; and
an outer flange, coupled to the obturator, adapted to remove the obturator from the shaft.

2. The chest tube introducer of claim 1, wherein the tubular shaft is L-shaped or curved.

3. The chest tube introducer of claim 1, wherein the gripping mechanism is two grip holes.

4. The chest tube introducer of claim 1, wherein the tubular shaft contains at least one aperture.

5. The chest tube introducer of claim 1, wherein the obturator is notched at its distal end.

6. The chest tube introducer of claim 1, wherein the gripping mechanism includes one or more directional arrows.

7. A method of introducing a chest tube into a pleural cavity, the method comprising:
inserting a tubular shaft with an obturator removably coupled to a distal end of the shaft into a pleural cavity,
removing the obturator from the tubular shaft, and
threading the chest tube through the shaft,
wherein the tubular shaft is coupled at a proximal end of the shaft with a gripping mechanism.

8. The method of claim 7, wherein the tubular shaft is curved or L-shaped.

9. The method of claim 7, wherein the gripping mechanism has one or more directional arrows depicting direction of the tubular shaft.

10. The method of claim 7, wherein obturator comprises a flat outer flange.

11. The method of claim 7, wherein the tubular shaft contains at least one aperture.
